(19) United States
(12) Patent Application Publication
(10) Pub. No.: US 2025/0177455 A1
(43) Pub. Date: Jun. 5, 2025

(54) NOVEL THERAPEUTIC COMPRISING A SECRETOME FROM MESENCHYMAL STEM CELLS OF UMBILICORD TISSUE OR WHARTON'S JELLY

(71) Applicant: Celligenics PTE LTD., Singapore (SG)

(72) Inventors: Aimin Yeo, Singapore (SG); Pamela Mok, Singapore (SG); Sixun Chen, Singapore (SG); Yi Bing Aw, Singapore (SG)

(73) Assignee: Celligenics PTE LTD., Singapore (SG)

(21) Appl. No.: 18/715,634

(22) PCT Filed: Nov. 25, 2022
(86) PCT No.: PCT/SG2022/050858

§ 371 Date: May 31, 2024

(30) Foreign Application Priority Data
Dec. 2, 2021 (GB) 2117430.5
Mar. 4, 2022 (GB) 2203017.5

(51) Int. Cl. A61K 35/51; A61K 38/00; A61K 38/17; A61K 38/18; A61K 38/19; A61K 38/20; A61K 38/48; A61P 17/02; C12N 5/0775

(52) U.S. Cl. CPC A61K 35/51; A61K 38/005; A61K 38/1774; A61K 38/1793; A61K 38/18; A61K 38/1833; A61K 38/1891; A61K 38/193; A61K 38/195; A61K 38/204; A61K 38/2053; A61K 38/2093; A61K 38/4886; A61P 17/02; C12N 5/0665; C12N 2500/12; C12N 2500/42; C12N 2501/11; C12N 2501/115; C12N 2501/135; C12N 2501/15; C12N 2501/998

(57) ABSTRACT

The invention concerns a novel therapeutic comprising a secretome obtained from mesenchymal stem cells of umbilical cord tissue or of wharton's jelly tissue and a formulation or pharmaceutical composition including same; ideally, but not exclusively, for use in treating a wound; a method for making said therapeutic; and a method of treating a wound using said therapeutic.

### FIELD OF THE INVENTION

The invention concerns a novel therapeutic and a formulation or pharmaceutical composition including same; ideally, but not exclusively, for use in treating a wound; a method for making said therapeutic; and a method of treating a wound using said therapeutic.

### BACKGROUND OF THE INVENTION

A wound is usually an injury where skin is torn, cut, or punctured (an open wound), or where a blunt force trauma causes a contusion (a closed wound). In either case, the epidermis of the skin is damaged.

Wounds can be classified as: clean—made under sterile conditions where there are no organisms present, and the skin is likely to heal without complications; contaminated—usually resulting from accidental injury and there are pathogenic organisms and foreign bodies in the wound; infected—the wound has pathogenic organisms present and multiplying and exhibiting clinical signs of infection; and colonized—a chronic situation, containing pathogenic organisms, difficult to heal (e.g. bedsore).

Further, wounds can be classified according to whether they are open or closed.

Open wounds are classified according to the object that caused the wound: incised wounds—caused by a clean, sharp-edged object such as a knife, razor, or glass splinter; lacerations—irregular tear-like wounds caused by some blunt trauma; abrasions (grazes)—superficial wounds in which the topmost layer of the skin (the epidermis) is scraped off; avulsions—where a body structure is forcibly detached from its normal point of insertion; puncture wounds—caused by an object puncturing the skin, such as a splinter, nail or needle; penetration wounds—caused by an object such as a knife entering and coming out from the skin; gunshot wounds—caused by a bullet or similar projectile driving into or through the body; and critical wounds—Including large burns that have been split.

Closed wounds have fewer categories, but are just as dangerous as open wounds: haematomas (or blood tumor)—caused by damage to a blood vessel that in turn causes blood to collect under the skin; haematomas that originate from internal blood vessel pathology are petechiae, purpura, and ecchymosis (the different classifications are based on size); haematomas that originate from an external source of trauma are contusions, also commonly called bruises; and crush injury—caused by a great or extreme amount of force applied over a long period of time.

Complications, such as bacterial infection of a wound, can impede the healing process and lead to life-threatening complications. For this reason it is important to ensure wounds heal quickly and effectively. Moreover, without rapid healing, the cost of wound care to health systems is an enormous drain on facilities.

The term “chronic wound” describes a wound that occurs in a patient who has physiologic impairments to healing. These impairments predispose cutaneous wounds to deviate from the characteristics of acute wound healing.

An estimated 3 to 6 million chronic skin ulcers occur in patients every year in the United States. The most common underlying impairments are venous reflux, pressure, and diabetes mellitus.

All wounds, particularly those refractory to healing, are candidates for wound healing therapy which promotes the acute wound response: thus compensating for missing or dysfunctional components present in the chronic wound.

The invention described herein concerns a new therapeutic for use particular, but not exclusively, in the healing of wounds, especially chronic wounds. This therapy works to activate signaling pathways of both epithelial and nonepithelial cells, and involves a myriad of different cytokines and growth factors. It is therefore a relatively complex but effective therapy.

### STATEMENTS OF THE INVENTION

According to a first aspect of the invention there is provided a therapeutic comprising a secretome obtained from mesenchymal stem cells of umbilical cord tissue or mesenchymal stem cells of wharton's jelly tissue.

Reference herein to wharton's jelly is a gelatinous substance within the umbilical cord, largely made up of mucopolysaccharides (hyaluronic acid and chondroitin sulfate). It acts as a mucous connective tissue containing some fibroblasts and macrophages, and is derived from extra-embryonic mesoderm.

In a preferred embodiment of the invention said secretome comprises or consists of the following components: Granulocyte colony-stimulating factor (G-CSF); Growth-regulated protein alpha (GRO-a); hepatocyte growth factor (HGF); Interleukin-6 (IL-6); Leukemia inhibitory factor (LIF); monocyte chemoattractant protein 3 (MCP-3); stem cell growth factor beta (SCGF-b); Tissue inhibitor of metalloproteinase 1 (TIMP-1); Matrix metalloproteinase 3 (MMP-3); Angiopoietin-like Protein 4 (ANGPTL4), Tumour necrosis factor receptor RI (TNF-RI) and CD63+ exosomes.

In a further preferred embodiment of the invention said secretome further comprises or consists of at least one of the following components, including any combination thereof: Interleukin-8 (IL-8); monocyte chemoattractant protein 1 (MCP-1); Macrophage migration inhibitory factor (MIF); Tissue inhibitor of metalloproteinase 2 (TIMP-2); Angiopoietin-1; Follistatin; Matrix metalloproteinase 1 (MMP-1); Matrix metalloproteinase 2 (MMP-2); and Matrix metalloproteinase 8 (MMP-8).

In yet a further preferred embodiment of the invention said components are present in the following relative amounts:

| Component | |
|---|---|
| G-CSF | 1.00 |
| GRO-a | 1.11 |
| HGF | 0.30 |
| IL-6 | 0.88 |
| IL-8 | 0.20 |
| LIF | 0.03 |
| MCP-1 | 0.02 |
| MCP-3 | 0.15 |
| MIF | 0.04 |
| SCGF-b | 10.99 |
| TIMP-1 | 0.70 |
| TIMP-2 | 0.29 |
| Angiopoietin-1 | 0.12 |
| Follistatin | 0.51 |
| MMP-1 | 0.05 |
| MMP-2 | 0.03 |
| MMP-3 | 0.15 |
| MMP-8 | 0.01 |
| TNF-RI | 0.04 |
| Angiopoietin-like | 0.79 |
| Protein 4 (ANGPTL4) | |

Reference herein to relative amounts is reference to the amount of each component having regard to a reference component i.e., granulocyte colony stimulating factor (G-CSF), which is designated as 1.0 at a concentration of 22166 μg/ml, thus the amount of each other component, in pg/ml, is divided by 22166 to derive a relative amount.

In yet a still further preferred embodiment said components are present in the following amounts:

| Component | pg/ml |
|---|---|
| G-CSF | 2392-27899 pg/ml, ideally 22166 pg/ml, |
| GRO-a | 6870-24583 pg/ml, ideally, 24583 pg/ml, |
| HGF | 5443-9709 pg/ml, ideally, 6653 pg/ml, |
| IL-6 | 2059-29353 pg/ml, ideally, 19404 pg/ml, |
| IL-8 | 575-7697 pg/ml, ideally, 4436 pg/ml, |
| LIF | 256-810 pg/ml, ideally, 655 pg/ml, |
| MCP-1 | 431-2204 pg/ml, ideally, 487 pg/ml, |
| MCP-3 | 637-4120 pg/ml, ideally, 3268 pg/ml, |
| MIF | 695-2700 pg/ml, ideally, 816 pg/ml, |
| SCGF-b | 144354-436153 pg/ml, ideally, 243596 pg/ml, |
| TIMP-1 | 1628-15579 pg/ml, ideally, 15579 pg/ml, |
| TIMP-2 | 3623-10543 pg/ml, ideally, 6391 pg/ml, |
| Angiopoietin-1 | 1996-4932 pg/ml, ideally, 2667 pg/ml, |
| Follistatin | 5062-17984 pg/ml, ideally, 11401 pg/ml, |
| MMP-1 | 169-1919 pg/ml, ideally, 1065 pg/ml, |
| MMP-2 | 565-829 pg/ml, ideally, 761 pg/ml, |
| MMP-3 | 637-4120 pg/ml, ideally, 3268 pg/ml, |
| MMP-8 | 24-209 pg/ml, ideally, 122 pg/ml, |
| TNF-RI | 256-1193 pg/ml, ideally, 830 pg/ml, |
| Angiopoietin-like | 7765-26807 pg/ml, ideally, 17527 pg/ml |
| Protein 4/ANGPTL4 | |

In yet a further preferred embodiment said therapeutic contains CD63+ exosomes in an amount that is about 2-30 μg/ml, ideally, 5-20 μg/ml.

In yet another embodiment of the invention said components are present in either the above relative amounts or in the above concentration amounts.

Reference herein to CD63 is reference to an antigen that is a protein and, in humans, is encoded by the CD63 gene. CD63 is mainly associated with membranes of intracellular vesicles.

According to a further aspect of the invention there is provided a method for making a therapeutic comprising a secretome obtained from mesenchymal stem cells of umbilical cord tissue or mesenchymal stem cells of wharton's jelly tissue wherein the method comprises:
1. culturing mesenchymal stem cells of the umbilical cord or mesenchymal stem cells of wharton's jelly to confluence;
2. removing growth medium and washing the cell layer with an isotonic salt solution;
3. incubating the cells with a growth factor free medium; then
4. incubating the cells with a growth factor medium; and
5. collecting the cell secretome from said cells.

In a preferred method of the invention steps 3 and/or 4 may be repeated once or more than once and step 5 may be performed after step 3 and/or step 4.

In yet a further preferred method, steps 3 and 4 may be reversed so that incubating with a growth factor medium proceeds incubating with a growth factor free medium.

In the above method, preferably said isotonic salt solution is phosphate buffered saline. This step is performed to remove growth medium components.

In the above method, preferably said growth factor free medium comprises a basal medium (e.g. DMEM) containing 0.01 to 0.5% human serum albumin or recombinant human serum albumin, 50-500 μM sodium ascorbyl phosphate, and pen-strep-glutamax-NEAA.

In the above method, preferably said growth factor medium comprises comprises a basal medium (e.g. DMEM) containing 0.1-21U FGF2 and/or EGF and/or TGF beta and/or PDGF, 0.01 to 0.5% human serum albumin or recombinant human serum albumin, 50-500 μM sodium ascorbyl phosphate, and pen-strep-glutamax-NEAA.

In the above method steps 3-6 may be repeated for a number of cycles. Ideally, in total, said cells are in culture/incubated for 2 to 14 days.

In yet a further preferred method of the invention the collected secretome is sterile filtered and concentrated via ultrafiltration. This concentrated secretome is pooled to give the final secretome used in downstream applications.

Yet more preferably still, the secretome is stabilized by adding 0.1-1% carrier protein, and/or 0.5-10 mM sodium ascorbyl phosphate, and/or 0.5-20% sucrose, and/or phosphate buffer.

The secretome may be frozen or lyophilized for long-term storage.

According to a further aspect of the invention there is provided a novel therapeutic comprising the secretome obtained by the above method.

According to a yet further aspect of the invention there is provided a method of treating a wound comprising administering the afore therapeutic to the wound.

According to a yet further aspect of the invention there is provided the therapeutic of the invention, optionally produced or obtained using the method of the invention, for use in wound treatment.

According to a yet further aspect of the invention there is provided the use of the therapeutic of the invention, optionally produced or obtained using the method of the invention, in the manufacture of a medicament for treating wounds.

In the claims which follow and in the preceding description of the invention, except where the context requires otherwise due to express language or necessary implication, the word “comprise”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

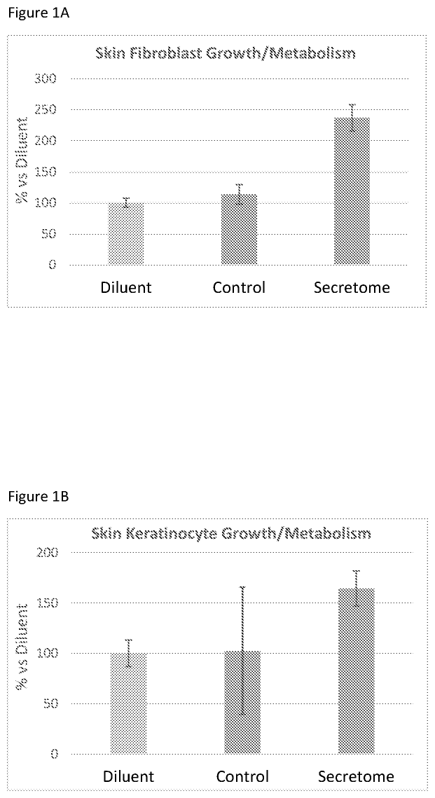

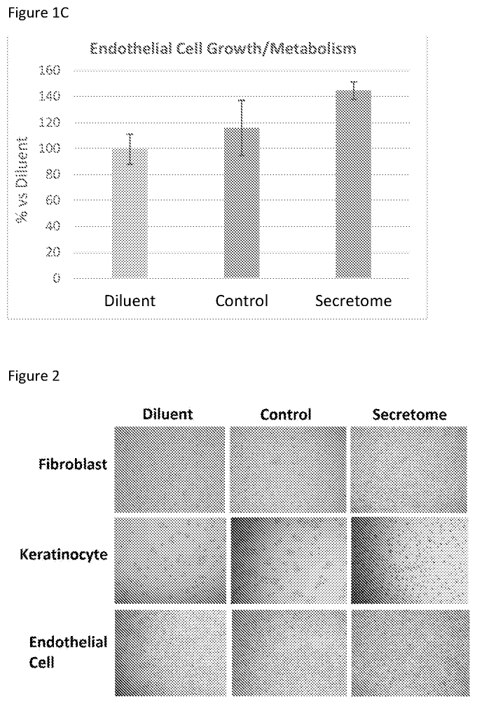

FIG. 1. Shows the effectiveness of the secretome on cell growth and metabolism, using human skin fibroblasts, human keratinocytes, and human endothelial cells. Cells were treated with assay diluent (untreated), un-conditioned medium (control), or secretome for 3 (endothelial cells), 4 (keratinocytes), or 5 (fibroblast) days. Cell growth and metabolism is shown for human skin fibroblasts (1A), human keratinocytes (1B), and human endothelial cells (1C).

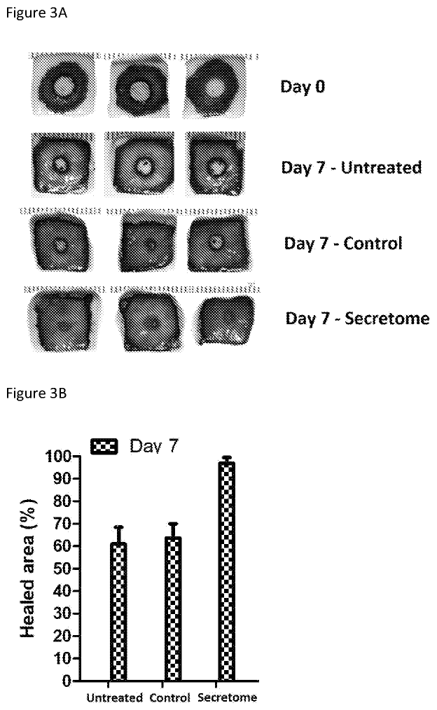

FIG. 2. Shows the effectiveness of the secretome on cell growth and metabolism, using human skin fibroblasts, human keratinocytes, and human endothelial cells. Cells were treated with assay diluent (untreated), un-conditioned medium (control), or secretome for 3 (endothelial cells), 4 (keratinocytes), or 5 (fibroblast) days; cell growth is demonstrated by staining the cells with safranin O.

FIG. 3. Shows the effect of the secretome on wound healing. De-epidermised human skin equivalent wounds were left untreated, treated with un-conditioned medium (control), or secretome for 3 days. On day 7, wound closure was assessed visually by MTT staining (3A), and quantitatively using the images in FIG. 3A (3B).

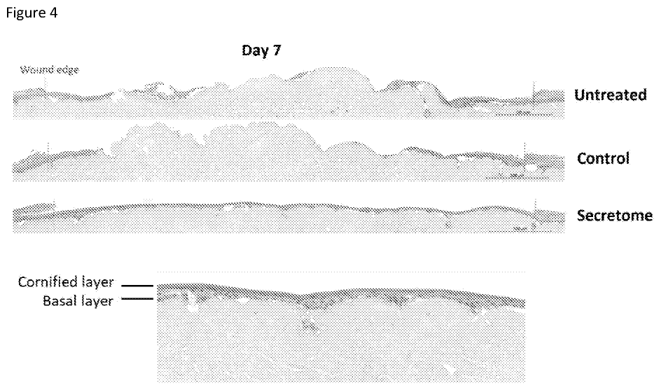

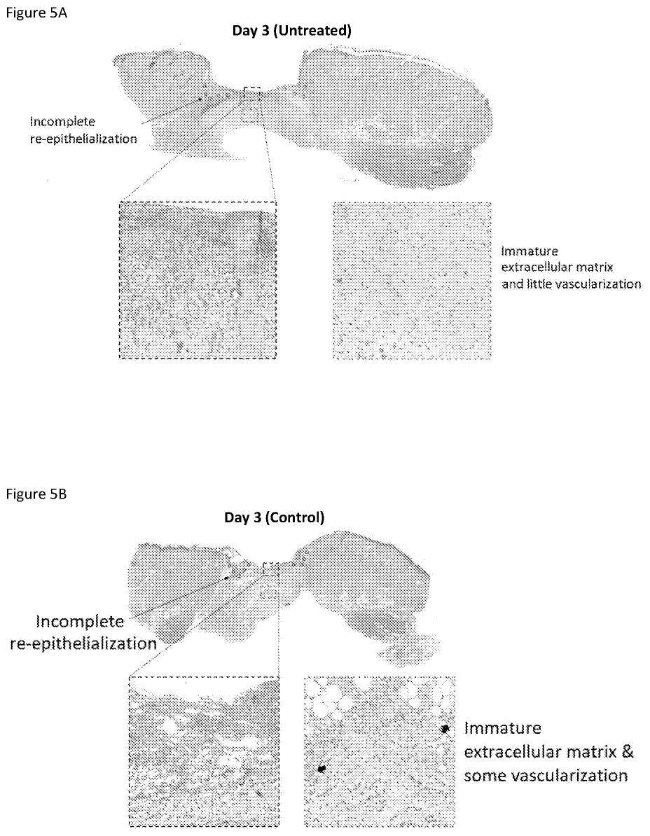

FIG. 4. Shows the effect of the secretome on wound healing. De-epidermised human skin equivalent wounds were left untreated, treated with un-conditioned medium (control), or secretome for 3 days. On day 7, wound closure was assessed visually by H&E staining after sectioning. Red vertical lines indicate original wound edge at Day 0. Secretome promotes keratinocyte proliferation & migration (faster wound bed closure) compared to control (P<0.05). Control (un-conditioned medium) show no effect vs untreated wounds (P>0.05).

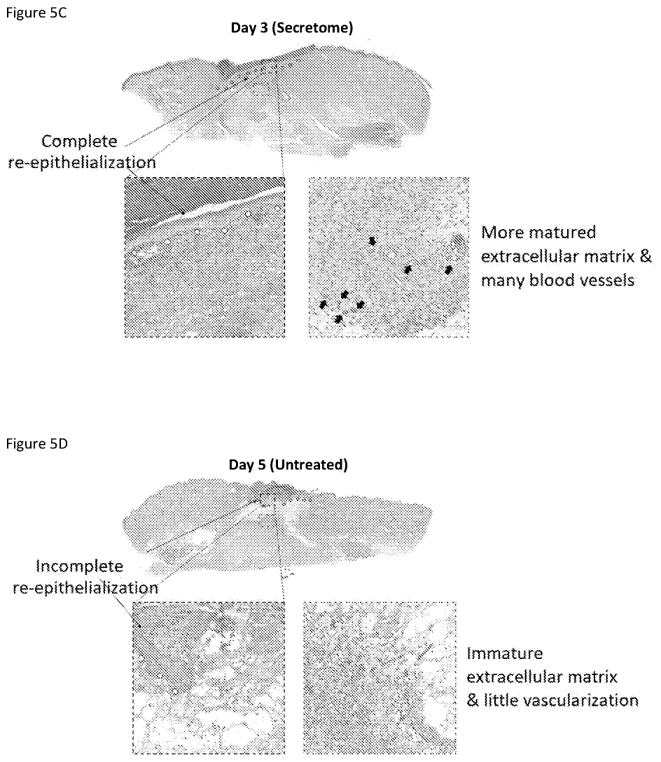

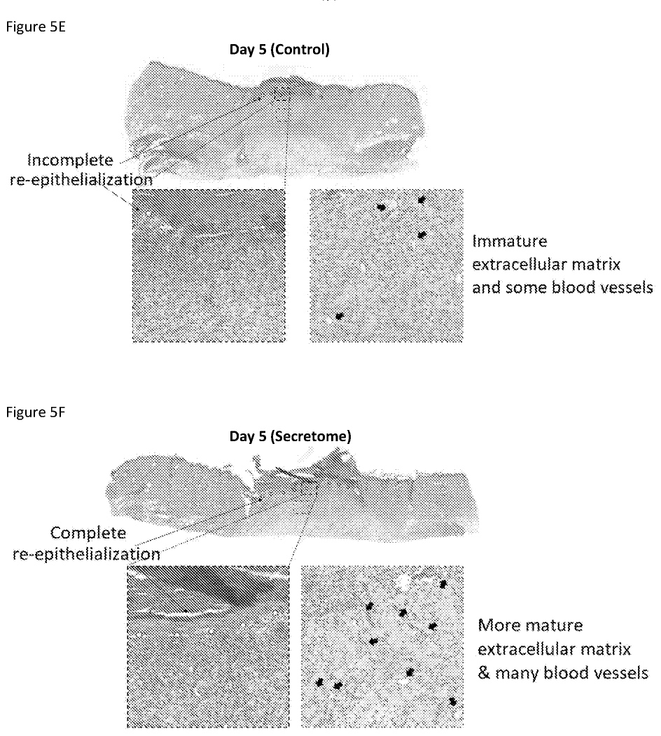

FIG. 5A. Shows the effect of the secretome on wound healing in vivo. Full thickness wounds were created in rats. Wounds were left untreated (5A & 5D), treated with un-conditioned medium (control 5B & 5E), or secretome (5C & 5F) for 3 days. On days 3 (5A-C) and 5 (5D-F), wound closure and wound bed analysis were assessed by H&E staining after sectioning.

### METHODS AND MATERIALS

### Stem Cells Secretome Production

1. Celligenics stem cells are cultured to 95-100% confluency.
2. Growth medium is removed and the cell layer is washed twice with an isotonic salt solution e.g. phosphate buffered saline to remove growth medium components.
3. Cells are then incubated with a growth factor free medium comprising a basal medium (e.g. DMEM) containing 0.01 to 0.5% human serum albumin or recombinant human serum albumin, 50-500 μM sodium ascorbyl phosphate, and pen-strep-glutamax-NEAA or a growth factor medium comprising a basal medium (e.g. DMEM) containing 0.1-21U FGF2 and/or EGF and/or TGF beta and/or PDGF, 0.01 to 0.5% human serum albumin or recombinant human serum albumin, 50-500 μM sodium ascorbyl phosphate, and pen-strep-glutamax-NEAA,
4. After e.g. 2 days, the medium (Day 2 secretome) is collected and step 3 is repeated.
5. Steps 3 and 4 can be repeated for several cycles to collect the secretome.

### Stem Cells Secretome Post-Production Processing

1. The collected secretome from above is sterile filtered and concentrated via ultrafiltration. The concentrated secretome from the collection days is pooled to give the final secretome used in downstream applications.
2. The secretome is stabilized before freezing or lyophilization by adding 0.1-1% carrier protein, and/or 0.5-10 mM sodium ascorbyl phosphate, and/or 0.5-20% sucrose, and/or phosphate buffer.

### Stem Cells Secretome Characterization

The collected secretome has been analysed and the active components comprises growth factors, cytokines, enzymes, signalling molecules, matrix proteins, and extracellular vesicles. The quantities listed below have been normalized and averaged to the amount that would be present in the secretome when it is first collected. The secretome also contains CD63+ exosomes (˜2-30 μg/ml).

| Component | pg/ml |
|---|---|
| G-CSF | 22166 |
| GRO-a | 24583 |
| HGF | 6653 |
| IL-6 | 19404 |
| IL-8 | 4438 |
| LIF | 655 |
| MCP-1 | 487 |
| MCP-3 | 3268 |
| MIF | 816 |
| SCGF-b | 243596 |
| TIMP-1 | 15579 |
| TIMP-2 | 6391 |
| Angiopoietin-1 | 2667 |
| Follistatin | 11401 |
| MMP-1 | 1065 |
| MMP-2 | 761 |
| MMP-3 | 3268 |
| MMP-8 | 122 |
| TNF-RI | 830 |
| Angiopoietin-like | 17527 |
| Protein 4/ANGPTL4 | |

### Use of Secretome for Wound Healing

### In-Vitro: 2D Cell-Based Assays

Aims & Methods: To assess the effect of the above secretome on cell growth and metabolism. Human skin fibroblasts, Human keratinocytes, and Human endothelial cells were seeded in 96-well plates. Cells were treated with assay diluent (untreated), un-conditioned medium (control), or secretome for 5 (fibroblast), 4 (keratinocytes), or 3 (endothelial cells) days. Cell growth and metabolism were assessed by staining with CCK8 reagent, subsequently reading the Absorbance at 450 nm, and referencing to a cell standard. Evidence of cell growth was further demonstrated by staining the cells with safranin O.

### In-Vitro: 3D Skin Model

Aims & Methods: To assess the effect of the secretome on wound healing, de-epidermised human skin equivalents were constructed. Wounds were left untreated, treated with un-conditioned medium (control), or secretome for 3 days. On day 7, wound closure was assessed by MTT staining, and H&E staining after sectioning.

### In-Vivo: Rat Wound Model

Aims & Methods: To assess potential effect of secretome on wound healing, full thickness wounds were created in rats. Wounds were left untreated, treated with un-conditioned medium (control), or secretome for 3 days. On days 3 and 5, wound closure and wound bed analysis were assessed by H&E staining after sectioning.

### Results

### Stem Cells Secretome Application

The secretome has been tested for wound healing properties in a variety of assays. The secretome promotes proliferation in skin fibroblasts, keratinocytes, and endothelial cells; promotes collagen production.

The secretome also promotes keratinocyte proliferation and differentiation in de-epidermised dermis human skin equivalents.

In animal models, the secretome promotes faster wound closure, angiogenesis, and matrix deposition/maturation.

FIGS. 1 and 2 show the secretome promotes human skin fibroblast, keratinocyte, and endothelial cell growth and metabolism compared to control (P<0.05). The control (un-conditioned medium) showed no effect vs cells untreated with secretome (P>0.05). In FIG. 2 the secretome treated panel shows more growth, implying increased metabolism.

We assessed the effect of the secretome on wound healing, using a de-epidermised human skin equivalent. FIGS. 3 and 4 show the effect of the secretome on wound healing.

In FIG. 3 it can be seen that wound closure is promoted by use of the secretome. The control has no effect.

In FIG. 4 images of wounds are shown. Red vertical lines indicate original wound edge for each wound at Day 0. The wound treated with secretome has been closed, it has a cornified layer and a basal layer spanning the wound, thus, it can be seen that the secretome has promoted keratinocyte proliferation & migration (faster wound bed closure) compared to control (P<0.05). Control (un-conditioned medium) shows no effect vs wounds untreated with secretome (P>0.05). The secretome promotes keratinocyte differentiation (epidermal stratification).

FIG. 5 charts wound healing in a rat model on days 3 and 5 after control or secretome treatment. The yellow dots trace the epithelial tongue and the black arrows indicate blood vessel formation. The secretome accelerates re-epithelialization, vascularization, and extracellular matrix maturation compared to no treatment or treatment with un-conditioned medium.

Taken together, the data clearly shows the regenerative effect of the secretome which can be used, advantageously, to treat wounds.

TABLE 1
Secretome of the invention showing the amounts of the
components in the secretome, both in terms of range and
average andwith reference to either SCGFb or G-CSF.

| | Low | High | Mean | vs SCGfb | vs G-CSF |
|---|---|---|---|---|---|
| IL-6 | 2059 | 29353 | 19404 | 0.080 | 0.88 |
| MCP-3 | 637 | 4120 | 3268 | 0.013 | 0.15 |
| LIF | 256 | 810 | 655 | 0.003 | 0.03 |
| MIF | 695 | 2700 | 816 | 0.003 | 0.04 |
| MCP-1 | 431 | 2204 | 487 | 0.002 | 0.02 |
| IL-8 | 575 | 7697 | 4438 | 0.018 | 0.20 |
| G-CSF | 2392 | 27899 | 22166 | 0.091 | 1.00 |
| GRO-a | 6870 | 24583 | 24583 | 0.101 | 1.11 |
| HGF | 5443 | 9709 | 6653 | 0.027 | 0.30 |
| SCGF-b | 144354 | 436153 | 243596 | 1.000 | 10.99 |
| TIMP-1 | 1628 | 15579 | 15579 | 0.064 | 0.70 |
| TIMP-2 | 3623 | 10543 | 6391 | 0.026 | 0.29 |
| MMP-2 | 565 | 829 | 761 | 0.003 | 0.03 |
| MMP-3 | 637 | 4120 | 3268 | 0.013 | 0.15 |
| TNF-RI | 256 | 1193 | 830 | 0.003 | 0.04 |
| Follistatin | 5062 | 17984 | 11401 | 0.047 | 0.51 |
| MMP-1 | 169 | 1919 | 1065 | 0.004 | 0.05 |
| MMP-8 | 24 | 209 | 122 | 0.001 | 0.01 |
| Angiopoietin-1 | 1996 | 4932 | 2667 | 0.011 | 0.12 |
| Angiopoietin-like | 7765 | 26807 | 17527 | 0.072 | 0.79 |
| Protein 4/ANGPTL4 | | | | | |

1. A therapeutic comprising a secretome obtained from mesenchymal stem cells of umbilical cord tissue or mesenchymal stem cells of wharton's jelly tissue wherein the secretome comprises or consists of the following components: Granulocyte colony-stimulating factor (G-CSF); Growth-regulated protein alpha (GRO-a); hepatocyte growth factor (HGF); Interleukin-6 (IL-6); Leukemia inhibitory factor (LIF); monocyte chemoattractant protein 3 (MCP-3); stem cell growth factor beta (SCGF-b); Tissue inhibitor of metalloproteinase 1(TIMP-1); Matrix metalloproteinase 3 (MMP-3); Angiopoietin-like Protein 4 (ANGPTL4) Tumour necrosis factor receptor RI (TNF-RI) and CD63+ exosomes.

2. The therapeutic according to claim 1 wherein said secretome comprises or consists of: at least one of the following components, including any combination thereof: Interleukin-8 (IL-8); monocyte chemoattractant protein 1 (MCP-1); Macrophage migration inhibitory factor (MIF); Tissue inhibitor of metalloproteinases 2 (TIMP-2); Angiopoietin-1; Follistatin; Matrix metalloproteinase 1 (MMP-1); Matrix metalloproteinase 2 (MMP-2); and Matrix metalloproteinase 8 (MMP-8).

3. The therapeutic according to claim 1 wherein said secretome comprises or consists of the said components in the following relative amounts with respect to the amount of G-CSF:

| Component | Relative Amount |
|---|---|
| G-CSF | 1.00 |
| GRO-a | 1.11 |
| HGF | 0.30 |
| IL-6 | 0.88 |
| IL-8 | 0.20 |
| LIF | 0.03 |
| MCP-1 | 0.02 |
| MCP-3 | 0.15 |
| MIF | 0.04 |
| SCGF-b | 10.99 |
| TIMP-1 | 0.7 |
| TIMP-2 | 0.29 |
| Angiopoietin-1 | 0.12 |
| Follistatin | 0.51 |
| MMP-1 | 0.05 |
| MMP-2 | 0.03 |
| MMP-3 | 0.15 |
| MMP-8 | 0.01 |
| TNF-RI | 0.04 |
| Angiopoietin-like | 0.79 |
| Protein 4/ANGPTL4 | |

4. The therapeutic according to claim 1 wherein said secretome comprises or consists of: the components in the following amounts:

| Component | pg/ml | |
|---|---|---|
| G-CSF | 2392-27899 | pg/ml |
| GRO-a | 6870-24583 | pg/ml |
| HGF | 5443-9709 | pg/ml |
| IL-6 | 2059-29353 | pg/ml |
| IL-8 | 575-7697 | pg/ml |
| LIF | 256-810 | pg/ml |
| MCP-1 | 431-2204 | pg/ml |
| MCP-3 | 637-4120 | pg/ml |
| MIF | 695-2700 | pg/ml |
| SCGF-b | 144354-436153 | pg/ml |
| TIMP-1 | 1628-15579 | pg/ml |
| TIMP-2 | 3623-10543 | pg/ml |
| Angiopoietin-1 | 1996-4932 | pg/ml |
| Follistatin | 5062-17984 | pg/ml |
| MMP-1 | 169-1919 | pg/ml |
| MMP-2 | 565-829 | pg/ml |
| MMP-3 | 637-4120 | pg/ml |
| MMP-8 | 24-209 | pg/ml |
| TNF-RI | 256-1193 | pg/ml |
| Angiopoietin-like | 7765-26807 | pg/ml |

Protein 4/ANGPTL4

5. The therapeutic according to claim 4 wherein the amount of each component is present in the following amount

| Component | pg/ml |
|---|---|
| G-CSF | 22166 |
| GRO-a | 24583 |
| HGF | 6653 |
| IL-6 | 19404 |
| IL-8 | 4438 |
| LIF | 655 |
| MCP-1 | 487 |
| MCP-3 | 3268 |
| MIF | 816 |
| SCGF-b | 243596 |
| TIMP-1 | 15579 |
| TIMP-2 | 6391 |
| Angiopoietin-1 | 2667 |
| Follistatin | 11401 |
| MMP-1 | 1065 |
| MMP-2 | 761 |
| MMP-3 | 3268 |
| MMP-8 | 122 |
| TNF-RI | 830 |
| Angiopoietin-like | 17527 |
| Protein 4/ANGPTL4 | |

6. The therapeutic according to claim 1 wherein said CD63+ exosomes are present in an amount that is 2-30 μg/ml or 5-20 μg/ml.

7. A method for making a therapeutic comprising a secretome obtained from mesenchymal stem cells of umbilical cord tissue or mesenchymal stem cells of wharton's jelly tissue wherein the method comprises:
i) culturing mesenchymal stem cells of the umbilical cord or mesenchymal stem cells of wharton's jelly to confluence;
ii) removing growth medium and washing the cell layer with an isotonic salt solution;
iii) incubating the cells with a growth factor free medium; then
iv) incubating the cells with a growth factor medium; and
v) collecting the cell secretome from said cells.

8. The method according to claim 7 wherein steps iii) and/or iv) may be repeated once or more than once and/or step v) may be performed after step iii) and/or step iv).

9. The method according to claim 7 wherein steps iii) and iv) are reversed so that incubating with a growth factor medium proceeds incubating with a growth factor free medium.

10. The method according to claim 7 wherein said isotonic salt solution is phosphate buffered saline.

11. The method according to claim 7 wherein said growth factor free medium comprises a basal medium containing 0.01 to 0.5% human serum albumin or recombinant human serum albumin, 50-500 μM sodium ascorbyl phosphate, and pen-strep-glutamax-NEAA.

12. The method according to claim 7 wherein said growth factor medium comprises a basal medium containing 0.1-2IU FGF2 and/or EGF and/or TGF beta and/or PDGF, 0.01 to 0.5% human serum albumin or recombinant human serum albumin, 50-500 μM sodium ascorbyl phosphate, and pen-strep-glutamax-NEAA.

13. The method according to claim 7 wherein the secretome is stabilized by adding 0.1-1% carrier protein, and/or 0.5-10 mM sodium ascorbyl phosphate, and/or 0.5-20% sucrose, and/or phosphate buffer.

14. A therapeutic comprising the secretome obtained by the method according to claim 7.

15. The therapeutic according to claim 14, wherein the secretome comprises or consists of the following components: Granulocyte colony-stimulating factor (G-CSF); Growth-regulated protein alpha (GRO-a); hepatocyte growth factor (HGF); Interleukin-6 (IL-6); Leukemia inhibitory factor (LIF); monocyte chemoattractant protein 3 (MCP-3); stem cell growth factor beta (SCGF-b); Tissue inhibitor of metalloproteinase 1(TIMP-1); Matrix metalloproteinase 3 (MMP-3); Angiopoietin-like Protein 4 (ANGPTL4) Tumour necrosis factor receptor RI (TNF-RI) and CD63+ exosomes.

16. A method of treating a wound comprising administering the therapeutic according to claim 1 to the wound, thereby treating the wound.

17. A method of treating a wound comprising administering the therapeutic according to claim 14 to the wound, thereby treating the wound.

18. A method of treating a wound comprising administering the therapeutic according to claim 15 to the wound, thereby treating the wound.
